(19) United States
(12) Patent Application Publication
(10) Pub. No.: US 2014/0015171 A1
(43) Pub. Date: Jan. 16, 2014

(54) STERILIZING METHOD AND APPARATUS FOR THE BLOW MOLDING OF CONTAINERS

(71) Applicants: Thomas Herold, Ahrensburg (DE); Harald Rieger, Hamburg (DE)

(72) Inventors: Thomas Herold, Ahrensburg (DE); Harald Rieger, Hamburg (DE)

(73) Assignee: KHS CORPOPLAST GMBH, Hamburg (DE)

(21) Appl. No.: 14/007,949

(22) PCT Filed: Feb. 17, 2012
(86) PCT No.: PCT/DE2012/000192

§ 371 Date: Sep. 26, 2013

(30) Foreign Application Priority Data
Mar. 29, 2011 (DE) 10 2011 016 448.0

(51) Int. Cl. B29C 49/42

(52) U.S. Cl. CPC B29C 49/4252
USPC 264/483; 425/174

(57) ABSTRACT

The method and the apparatus serve for sterilizing parisons made of a thermoplastic material which are provided for producing blow-molded containers. Sterilization is carried out using a plasma. A sterilizing device is provided, for this purpose, with a plasma generator. Sterilization is carried out following insertion of the parison into a blow-molding station or on a transfer wheel. The plasma is generated outside the parison and introduced into the parison.

The invention relates to a method of sterilizing parisons made of a thermoplastic material that are intended for producing blow-molded containers.

The invention furthermore relates to an apparatus for the blow-molding of containers that are sterile at least in areas, which apparatus comprises at least one blow molding station arranged on a carrier structure for the reforming of thermoplastic parisons into the containers.

A production of sterile, blow-molded containers typically takes place in such a manner that these containers are sterilized after their blow-molding and before being filled, using hydrogen peroxide or other chemicals. It is also already known to sterilize the parisons used as initial product in the blow-molding of containers, in particular the area of the inner surface of these parisons.

It is also already known that when carrying out a coating of containers using a plasma, sterilization effects occur. On account of the customarily provided contours in the area of the container walls, however, so-called shadings occur in such sterilizations that oppose a reliable sterilization.

A sterilization apparatus for parisons is described in WO 2009/026869 in which the sterilization is carried out using a plasma. The sterilization apparatus is arranged along a transport path of the parisons that extends from a supply apparatus for the parisons to a blow wheel.

In a container molding by the action of blowing pressure, parisons made of a thermoplastic material, for example, parisons made of PET (polyethylene terephthalate) are supplied inside a blow molding machine to different workstations. Typically, such a blow molding machine comprises a heating apparatus as well as a blowing apparatus in whose area the previously tempered parison is expanded by biaxial orientation to a container. The expansion takes place with the aid of compressed air that is introduced into the parison to be expanded. The technical course of the method in the case of such an expansion of the parison is explained in DE-OS 43 40 291.

The basic construction of a blowing station for the molding of containers is described in DE-OS 42 12 583. Possibilities for tempering the parisons are explained in DE-OS 23 52 926.

Inside the apparatus for blow molding the parisons as well as the blown containers can be transported with the aid of different handling devices. In particular the usage of transport mandrels onto which the parisons are inserted has proven itself. However, the parisons can also be handled with other carrying devices. The use of grippers for managing parisons and the use of expanding mandrels that can be introduced into a mouth area of the parison for holding it also belong to the available constructions.

A handling of containers using transfer wheels is described, for example, in DE-OS 199 06 438 in an arrangement of the transfer wheel between a blow wheel and an output section.

The already explained handling of the parisons takes place on the one hand in the so-called two-stage processes in which the parisons are first produced in an injection molding process, subsequently intermediately stored and only later conditioned as regards their temperature and blown up to a container. On the other hand a usage takes place in the so-called one-stage processes in which the parisons are appropriately tempered immediately after their production with injection molding technology and a sufficient solidification and subsequently blown up.

As regards the blow molding stations used, different designs are known. In blow molding stations that are arranged on rotating transport wheels a book-like ability to fold the mold carriers open is frequently found. However, it is also possible to use mold carriers that are guided so that they shift relative to each other or in another manner. In the case of stationary blow molding stations, that are in particular suitable for receiving several cavities for the container molding, plates that are typically arranged parallel to each other are used as mold carriers.

The present invention has the problem of improving a process of the initially cited type in such a manner that a reliable sterilization can be carried out in a simple manner.

This problem is solved in accordance with the invention in that the sterilization is carried out after the parison has been set into a blow molding station by using a plasma, whereby the plasma is produced outside of the parison and is introduced into the parison.

Another problem of the present invention is to construct an apparatus of the initially cited type in such a manner that an effective sterilization can be carried out with low expense.

This problem is solved in accordance with the invention in that the blow molding station is connected to at least one plasma generator that produces the plasma with a spatial distance to the parison, whereby the plasma generator is coupled via at least one flow path to an inner chamber of the blow molding station.

The sterilization of the parisons inside the blow molding station by using a plasma optimizes the solution of all requirements made regarding a reliable and effective sterilization in the production of blow-molded containers.

Alternatively, the possibility has also been considered of arranging the plasma generator in the area of a transfer wheel or transport wheel and of sterilizing the parison here from the inside and/or from the outside. This takes place alternatively to or as a supplement to the arrangement of the plasma generator in the area of the blow wheel.

In contrast to a sterilization of blown containers, a significantly smaller surface to be sterilized is present in the parisons. In contrast to a sterilization of parisons in the area of a heating or along a transport path of the parisons, additional structural units are eliminated. The use of the plasma for sterilization avoids the occurrence of residues of a sterilization agent in the blown containers.

The danger of a re-contamination is avoided by the sterilization of the parisons inside the blow molding station in contrast to a sterilization of the parisons in the area of a heating, and at the same time the advantage of a small surface to be sterilized is obtained. Thus, inside the course of the process of the blow molding of containers from the parisons the sterilization of the parison takes place in time at the last possible moment in time before the enlarging of the surface by the blowing procedure.

The process in accordance with the invention as well as the construction in accordance with the invention make possible an extremely compact structure, an economical carrying out of the sterilization with a very high assurance of sterilization and while extensively avoiding additional process times at the same time.

According to a typical embodiment it is conceived that an inner surface of the parisons is sterilized.

An economical production of the plasma is supported in that the plasma treatment is carried out at ambient pressure.

In order to make a compact construction available, it is helpful that the plasma is introduced by a stretching rod into the parison.

In order to avoid or reduce additional processing times for carrying out the sterilization, the possibility was conceived that the plasma flows out of the stretching rod during an insertion of the stretching rod into the parison in the direction of an inner wall of the parison.

An effective carrying out of the sterilization procedure is supported in that the plasma exits out of the stretching rod in the area of an end of the stretching rod extending into the parison.

It is provided for a sterilization that is complete in a circumferential direction that the plasma exits from an outflow nozzle of the stretching rod, whereby the outflow nozzle is designed like a slit at least in areas.

It proved to be advantageous for high-performance machines that a plasma generator generating the plasma is transported in common with the blow molding station in a revolving motion by a blow wheel.

The drawings schematically show exemplary embodiments of the invention, in which:

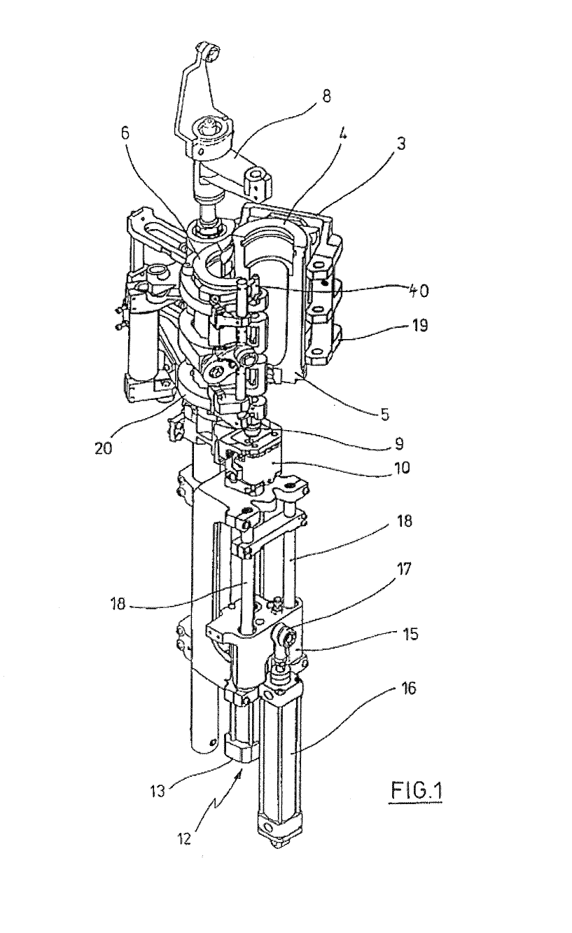

FIG. 1 shows a perspective view of a blow molding station for producing containers made from parisons,

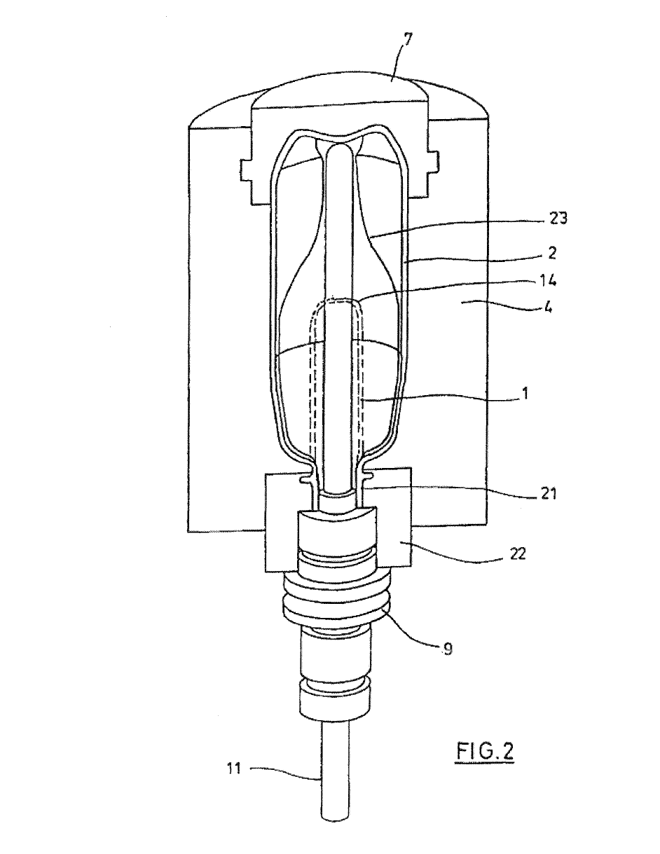

FIG. 2 shows a longitudinal section through a blow mold in which a parison is stretched and expanded,

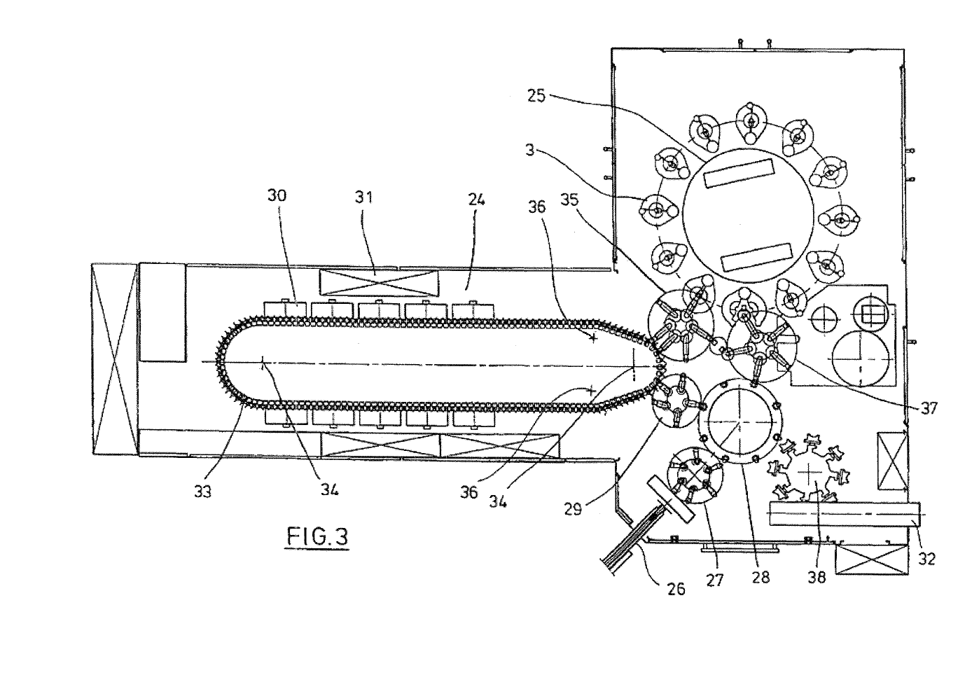

FIG. 3 shows a sketch for illustrating a basic construction of an apparatus for the blow molding of containers,

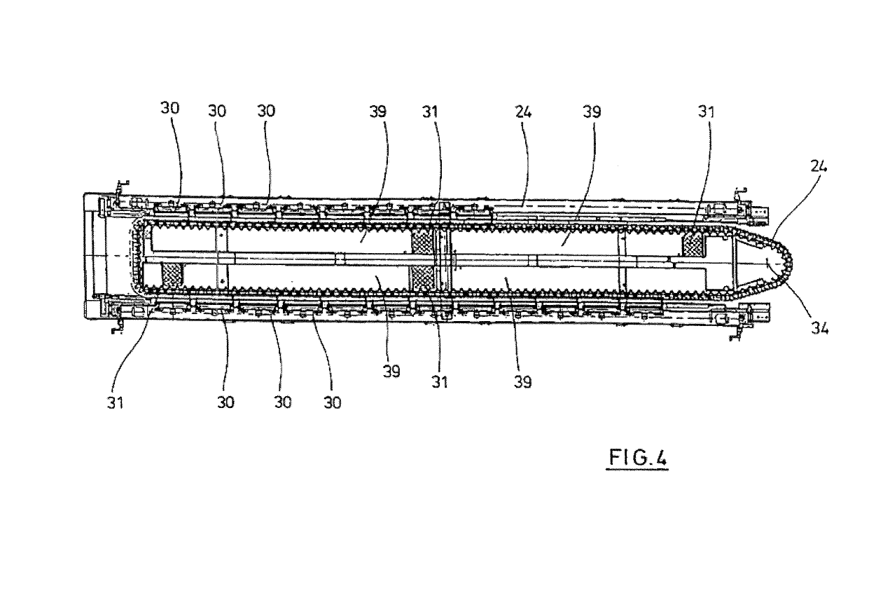

FIG. 4 shows a modified heating section with increased heating capacity,

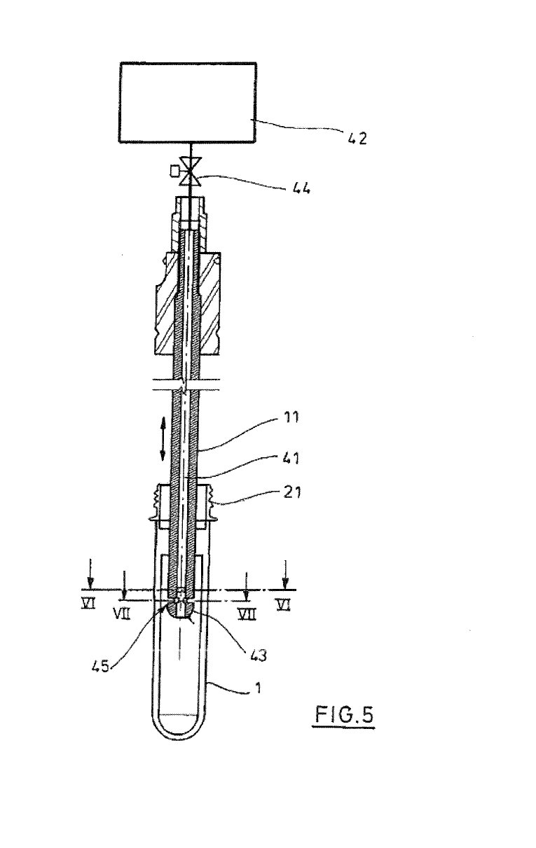

FIG. 5 shows a schematic longitudinal section through a parison into which a hollow stretching rod is inserted for supplying a plasma at the ambient pressure,

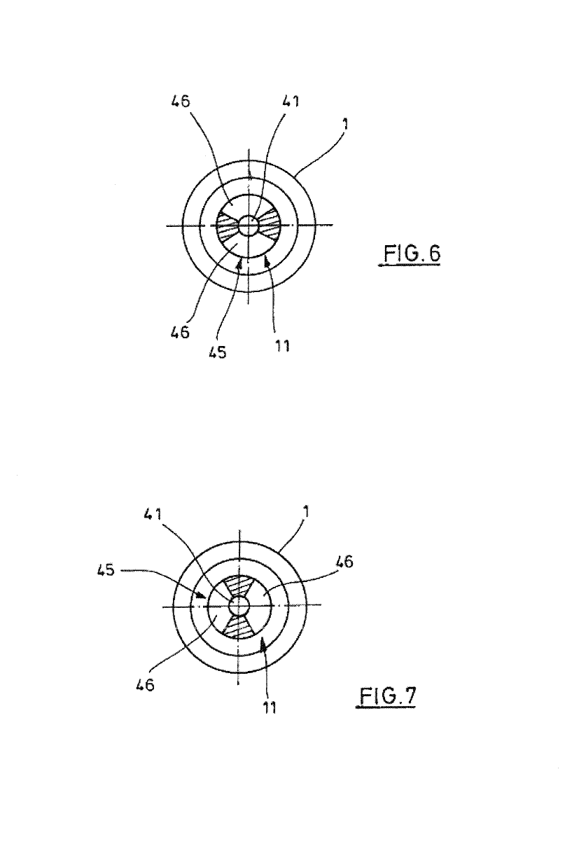

FIG. 6 shows a horizontal section through the stretching rod according to section line VI in FIG. 5, and

FIG. 7 shows a horizontal section through the stretching rod according to section line VII in FIG. 5.

In the following the basic construction of a blow molding machine will be described at first before an explanation of the detailed construction of the apparatus for sterilizing the parisons (1) by using a plasma as well as before an explanation of a concrete inclusion of a corresponding apparatus in a blow molding machine.

The basic construction of an apparatus for reforming parisons (1) into containers (2) is shown in FIG. 1 and in FIG. 2.

The apparatus for molding the container (2) consists substantially of a blow molding station (3) provided with a blow mold (4) into which a parison (1) can be inserted. The parison (1) can be an injection-molded part made of polyethylene terephthalate. In order to make it possible to insert the parison (1) into the blow mold (4) and to make possible a removal of the finished container (2) the blow mold (4) consists of mold halves (5, 6) and of a bottom part (7) that can be positioned by a lifting apparatus (8). The parison (1) can be held in the area of the blow molding station (3) by a transport mandrel (9) that runs through a plurality of treatment stations inside the apparatus jointly with the parison (1). However, it is also possible to insert the parison (1) directly into the blow mold (4), for example, by grippers or other handling means.

In order to make a compressed air supply line possible, a connection piston (10) is arranged underneath the transport mandrel (9) that supplies compressed air to the parison (1) and at the same time carries out a sealing relative to the transport mandrel (9). However, it is basically also conceivable to use fixed compressed air supply lines in a modified construction.

A stretching of the parison (1) takes place with the aid of a stretching rod (11) positioned by a cylinder (12). However, is basically also conceivable to perform a mechanical positioning of the stretching rod (11) with cam segments that are loaded by sensing rollers. The use of cam segments is especially advantageous if a plurality of blow molding stations (3) are arranged on a rotating blow wheel. A use of cylinders (12) is advantageous if stationarily arranged blow molding stations (3) are provided.

In the embodiment shown in FIG. 1 the stretching system is constructed in such a manner that a tandem arrangement of two cylinders (12) is made available. The stretching rod (11) is first moved by a primary cylinder (13) before the beginning of the actual stretching procedure into the area of a bottom (14) of the parison (1). During the actual stretching procedure the primary cylinder (13) is positioned with extended stretching rod together with a carriage (15) carrying the primary cylinder (13) by a secondary cylinder (16) or via a cam control. In particular, the secondary cylinder (16) is used with cam control in such a manner that an actual stretching position is given by a guide roller (17) that slides along a cam path during the carrying out of the stretching procedure. The guide roller (17) is pressed by the secondary cylinder (16) against the guide path. The carriage (15) slides along two guide elements (18).

After a closing of the mold halves (5, 6) arranged in the area of carriers (19, 20) a locking of the carriers (19, 20) takes place relative to one another with the aid of a locking device (40).

For the adaptation to different forms of a mouth section (21) of the parison (1) according to FIG. 2 the use of separate threaded inserts (22) in the area of the blow mold (4) is provided.

FIG. 2 also additionally shows, in addition to the blown container (2), the parison (1) sketched in dotted lines and schematically shows a developing container bubble (23).

FIG. 3 shows the basic construction of a blow molding machine, which is provided with a heating section (24) and a rotating blow wheel (25). Starting from an insertion (26) of a parison the parisons (1) are transported by transfer wheels (27, 28, 29) into the area of the heating section (24). Heating radiators (30) as well as blowers (31) are arranged along the heating section (24) in order to temper the parisons (1). After a sufficient tempering of the parisons (1) they are transferred to the blow wheel (25), in whose area the blow molding stations (3) are arranged. The containers (2) that are blown and finished are supplied by other transfer wheels to a discharge section (32).

In order to be able to reform a parison (1) into a container (2) in such a manner that the container (2) has material properties that ensure a long ability to use food, especially beverages, filled inside the container (2), special process steps must be maintained during the heating or orientation of the parisons (1). In addition, advantageous effects can be achieved by maintaining special dimensioning regulations.

Different plastics can be used as thermoplastic material. For example, PET, PEN or PP are capable of being used.

The expansion of the parison (1) during the orientation procedure takes place by the supplying of compressed air. The supplying of compressed air is divided into a pre-blowing phase in which gas, for example, compressed air, with a low pressure level is supplied, and into a following main blowing phase in which gas with a higher pressure level is supplied. During the pre-blowing phase compressed air with a pressure at an interval of 10 bar to 25 bar is typically used and during the main blowing phase compressed air with a pressure at an interval of 25 bar to 40 bar is supplied.

It can also be recognized from FIG. 3 that in the embodiment shown the heat section (24) is constructed from a plurality of rotating transport elements (33) that are arranged adjacent to one another in a chain-like manner and are guided along by deflection wheels (34). In particular, the concept is to mount a substantially rectangular basic contour by the chain-like arrangement. In the embodiment shown an individual, relatively large-dimensioned deflection wheel (34) is used in the area of the extension of the heating section (24) facing the transfer wheel (29) and a feed wheel (35) and to use two comparatively smaller-dimensioned deflection wheels (36) in the area of adjacent deflections. However, basically any other guides are conceivable.

In order to make possible the tightest possible arrangement of the transfer wheel (29) and of the feed wheel (35) relative to one another the arrangement shown proves to be especially advantageous since three deflection wheels (34, 36) are positioned in the area of the corresponding extension of the heating section (24) and, namely, the smaller deflection wheels (36) in the area of the transition to the linear courses of the heating section (24) and the larger deflection wheel (34) in the immediate transfer area to the transfer wheel (29) and to the feed wheel (35). For example, it is also possible to use a rotating heating wheel as an alternative to using chain-like transport elements (33).

After a finished blowing of the containers (2) they are guided out of the area of the blow molding station (3) by a removal wheel (37) and transported via the transfer wheel (28) and a discharge wheel (38) to the discharge section (32).

In the modified heating section (24) shown in FIG. 4, a larger amount of parisons (1) per time unit can be tempered by the greater number of heating radiators (30). The blowers (31) conduct cooling air here into the area of cooling air conduits (39) that are located opposite the associated heating radiators (30) and emit the cooling air via outflow openings. As a result of the arrangement of the outflow directions a direction of flow for the cooling air is realized substantially transversely to a transport direction of the parisons (1). The cooling air conduits (39) can make reflectors available for the heat radiation in the area of the heating radiators (30) and it is also possible to realize a cooling of the heating radiators (30) via the emitted cooling air.

FIG. 5 shows a longitudinal section through a parison (1) into which a stretching rod (11) is introduced that is constructed hollow at least in sections and that has an inner chamber (41). The inner chamber (41) is connected to a plasma generator (42) that is typically designed as a plasma jet. The plasma generator (42) generates a gaseous plasma by electrical discharge processes at a pressure that corresponds at least approximately to an ambient pressure and is therefore approximately 1 bar. Air taken from the surroundings is typically ionized using the electrical discharge. However, it is also basically conceivable to supply special process gases or to mix such process gases with ambient air.

The plasma generator (42) is connected in the exemplary embodiment according to FIG. 5 to an end of the stretching rod (11) which end is arranged facing away from a dome (43) that can be introduced into the parison (1). A connection line between inner chamber (41) of the stretching rod (11) and the plasma generator (42) can be closed by a valve (44). A closed valve (44) prevents in particular a flow of blow gas from the parison (1) and/or the container (2) in the direction of the plasma generator (42).

The stretching rod (11) comprises one or more outflow nozzles (45) from which the plasma exits in the direction of an inner wall of the parison (1). A loading of the inner wall of the parison (1) with the plasma causes a sterilization or at least a significant reduction of germs. The sterilization procedure is typically carried out during an introduction of the stretching rod (11) into the inner chamber (41). This minimizes or even avoids additional processing time.

An advance of the stretching rod (11) typically takes place in such a manner that an exposure time of the plasma on each area of the inner surface of the parison (1) is ensured for a time period of approximately 5 milliseconds. This results in a sterilization time of on the whole 15-150 milliseconds depending upon a size of the outflow nozzle (45). An introduction rate of the stretching rod (11) into the parison (1) during the carrying out of the sterilization procedure is approximately 0.1 to 2.0 m/sec. A rate in the range of 1.0 to 2.0 m/sec is preferred.

A distance between the outflow nozzle (45) and the inner wall of the parison (1) during the carrying out of the sterilization procedure is approximately 1.0 to 20 mm. This distance is selected in such a manner that the developing plasma flame can reach the inner surface of the parison (1) as a function of a flow rate of the plasma.

FIG. 6 and FIG. 7 show horizontal sections through the stretching rod (11) in the area of the outflow nozzle (45). It can be recognized that the outflow nozzle (45) consists of one or more slots (46). It was conceived in particular that at least two rows of slots (46) are arranged superposed over one another in a longitudinal direction of the stretching rod (11). FIG. 6 and FIG. 7 show an embodiment here in which each of these rows consists of two slots (46) separated from one another in a circumferential direction by a web. This construction ensures on the one hand a necessary mechanical rigidity and in addition it is ensured that plasma can exit from the inner chamber (41) of the stretching rod (11) along the entire circumferential area of the stretching rod (11).

According to a typical embodiment, the blow molding station (3), as well as the plasma generator (42), is arranged on a rotating blow wheel (45). Since the plasma generator (42) operates at ambient pressure and uses the ambient air as gas to be ionized, the plasma generator (42) requires only a supply of electrical energy. Since the latter is supplied in any case to the blow wheel (45), no additional supply connections are required.

According to a typical process course, the parisons (1) are inserted into the blow molding station (3) and before, during or after a closing of the mold halves (5, 6) the stretching rod (11) moves into the inner chamber (1) of the parison (1). During the moving in of the stretching rod (11) the plasma generator (42) generates the required plasma and the latter exits from the outflow nozzle (45) in order to carry out the sterilization procedure.

After an end of the sterilization procedure and a closing of the blow molding station (3) that took place at least up to then, the blow gas is supplied and the parison (1) is reformed into the container (2).

Thus, aside from the plasma generator (42) and the stretching rod (11), that is constructed hollow, no other structural elements are required for carrying out the sterilization procedure. The construction principle supports in particular the equipping of a standard blow molding machine with the sterilization device that takes place only when needed when this machine is required for special applications.

1. A method of sterilizing a parison made of a thermoplastic material that is intended for producing a blow-molded container, the method comprising:
setting the parison into a blow molding station; and
sterilizing the parison in the blow molding station using a plasma, wherein the plasma is produced outside of the parison and is introduced into the parison.

2. The method according to claim 1, wherein an inner surface of the parison is sterilized.

3. The method according to claim 1, wherein the plasma treatment is carried out at ambient pressure.

4. The method according to claim 1, wherein the plasma is introduced by a stretching rod inserted into the parison.

5. The method according to claim 4, wherein the plasma flows out of the stretching rod during an insertion of the stretching rod into the parison in a direction of an inner wall of the parison.

6. The method according to claim 4, wherein the plasma exits out of the stretching rod in the area of an end of the stretching rod extending into the parison.

7. The method according to claim 4, wherein the plasma exits from an outflow nozzle of the stretching rod, whereby the outflow nozzle configured as a slit at least in areas.

8. The method according to claim 1, wherein a plasma generator that generates the plasma is transported in common with the blow molding station in a revolving motion by a blow wheel.

9. An apparatus for the blow molding of containers comprising at least one blow molding station arranged on a carrier structure for the reforming of thermoplastic parisons into the containers, wherein at least one component of a rotating wheel of the apparatus is coupled to at least one plasma generator.

10. The apparatus according to claim 9, wherein the plasma generator is coupled via a flow path to an inner chamber of a stretching rod.

11. The apparatus according to claim 9, wherein the plasma generator is a plasma jet.

12. The apparatus according to claim 10, wherein the stretching rod comprises at least one outflow nozzle for the plasma.

13. The apparatus according to claim 12, wherein the outflow nozzle configured in a slotted manner at least in areas.

14. The apparatus according to claim 9, wherein the plasma generator and the blow molding station are arranged on a blow wheel capable of rotation.

15. The apparatus according to claim 9, wherein the blow molding station is connected to the at least one plasma generator such that the plasma generator produces plasma with a spatial distance to the parisons during sterilization, and wherein the plasma generator is coupled via at least one flow path to an inner chamber of the blow molding station.
